(19) United States
(12) Patent Application Publication
(10) Pub. No.: US 2007/0270877 A1
(43) Pub. Date: Nov. 22, 2007

(54) Drilling angle guide for use in orthopaedic surgery

(76) Inventor: SangDo Park, Philadelphia, PA (US)

Correspondence Address: SANGDO PARK, 2400 CHESTNUT ST., APT. 311, PHILADELPHIA, PA, 19103, US

(21) Appl. No.: 11/412,221

(22) Filed: Apr. 27, 2006

(51) Int. Cl. A61B 17/60; A61F 2/00

(52) U.S. Cl. 606/96

(57) ABSTRACT

An apparatus to guide drilling into the bone at the desired drilling angle from an entry point on the surface of the bone comprises rotationally linked members to allow the drill guide to rotate in two perpendicular axes to establish the drilling orientation. The drilling orientation is derived with respect to a reference structure on which the apparatus is mounted. After the drilling orientation is established, the appropriate drilling bit or guide wire is inserted into the drill guide, and the apparatus provides a means to translate the drill guide in two planes of motion to in order to coincide the tip of the drill bit or guide wire directly onto the entry point on the surface of the bone.

### REFERENCES CITED

U.S. Patent Documents

| | | |
|---|---|---|
| 4,907,577 | March 1990 | Wu |
| 6,120,511 | September 2000 | Chan |
| 6,342,056 | January 2002 | Mac-Thiong, et al. |

### BACKGROUND OF THE INVENTION

1. Technical Field

This apparatus guides drilling into the bone at the desired drilling angle from an entry point on the surface of the bone. This is of relevance to the field of orthopaedic surgery where screws and other implants need to be placed into the bone at the correct angle for structural and safety reasons.

2. Description of the Prior Art

Proper placement of the screws into the bone is an important but difficult part of orthopaedic surgery. The surgeon only sees the surface of the bone which he is drilling into and cannot directly visualize the path of drilling. The localization of entry point of drilling is not difficult with knowledge of basic anatomy and hence is not the problem. The difficulty lies in the angle of drilling from the entry point. If not properly drilled with the correct angle, the screw can penetrate out of the bone en route and injure adjacent soft tissue structures. It also would fail to perform its original function, which ranges from fracture fixation to structural support. Traditionally, the sum of surgical experience, anatomic knowledge, and gross visualization of patient position and nearby anatomic structures had guided the surgeon in orienting the drill. It should therefore come as no surprise that error in screw placement is frequently encountered in practice. Currently, intra-operative X-ray imaging is available to assist the surgeon in obtaining correct screw placement during every step of the process from aligning the drill bit onto the surface of the bone to checking the final position of the screw inside the bone. However, the disadvantage of this method is that frequent imaging is often needed as the drill is advanced into the bone and hence exposes both the patient and the surgeon to excessive radiation. Also, frequent imaging results in frequent changing of the direction of drilling and such changes within the bone can weaken the bone structurally. This also increases the operating time. Hence an apparatus and a method for bone drilling that minimize the use of intra-operative X-ray guidance and achieve correct screw placement in a single attempt without sacrificing accuracy, surgical time, and bone integrity are warranted.

This apparatus achieves the present task via exploiting the fact that the angles of bony processes in various planes (for instance, the angle between the neck of the femur and the shaft of the femur) are relatively constant throughout the population and are known in the orthopaedic literature. Moreover, bone imaging, whether via X-ray or MRI is always performed prior to orthopaedic procedures, and thus the precise angles of bony processes for the specific patient can be readily accessed and individually determined.

A particularly problematic area of screw insertion encountered by the orthopaedic surgeon is the pedicle of the vertebra. This is due to the fact that the angle of the pedicle with respect to the sagittal plane of the vertebra (i.e. the medial/lateral angulation) and the angle with respect to the axial plane of the vertebra (i.e. cephalad/caudal angulation) change with different levels (i.e. thoracic or lumbar) of vertebrae. This is complicated by the close proximity of important neural structures which leave little room for error in screw placement. As such, this is one area where such a guiding apparatus may be useful. Unfortunately, no such apparatus is in use in modern spine surgery despite the patents that exist (Wu U.S. Pat. No. 4,907,577; Mac-Thiong U.S. Pat. No. 6,342,056). This may be due to the fact that these devices are rather bulky, cumbersome, and complicated with multiple parts and may not function as exactly designed in the actual operative setting.

### BRIEF SUMMARY OF THE INVENTION

This apparatus seeks to guide drilling into the bone at the desired angle of entry from an entry point on the surface of the bone. The desired drilling angle is obtained from the knowledge of anatomy and/or pre-operative imaging such as X-ray and MRI and is made with respect to the reference structure to which the apparatus is mounted. The apparatus allows for selection of the appropriate angles of entry in two perpendicular planes via manipulation of the rotationally linked members. After that, the sliding mechanism of the apparatus localizes the drill guide directly onto the entry point on the surface of the bone. In doing so, this apparatus seeks to minimize intra-operative X-ray use, potential complications of poorly placed screws, and gain accepted use during routine orthopaedic surgeries especially during placement of vertebral pedicle screws.

### BRIEF DESCRIPTION OF THE DRAWINGS

For a fuller understanding of the nature and objects of the invention, reference should be had to the following detailed description taken in conjunction with the accompanying drawings in which:

FIG. 1 is a side view of one embodiment of the apparatus with cross-section through the cylindrical slot and the loop portion of the body.

FIG. 2 is an end-on cross-sectional view of the drill guide and the barrel.

FIG. 3 is a view of the apparatus from the top.

FIG. 4 is a side view of a vertebra.

FIG. 5 is an axial view of a vertebra.

FIG. 6 is posterior view of a vertebra with the apparatus mounted.

### DETAILED DESCRIPTION OF THE PREFERRED EMBODIMENTS

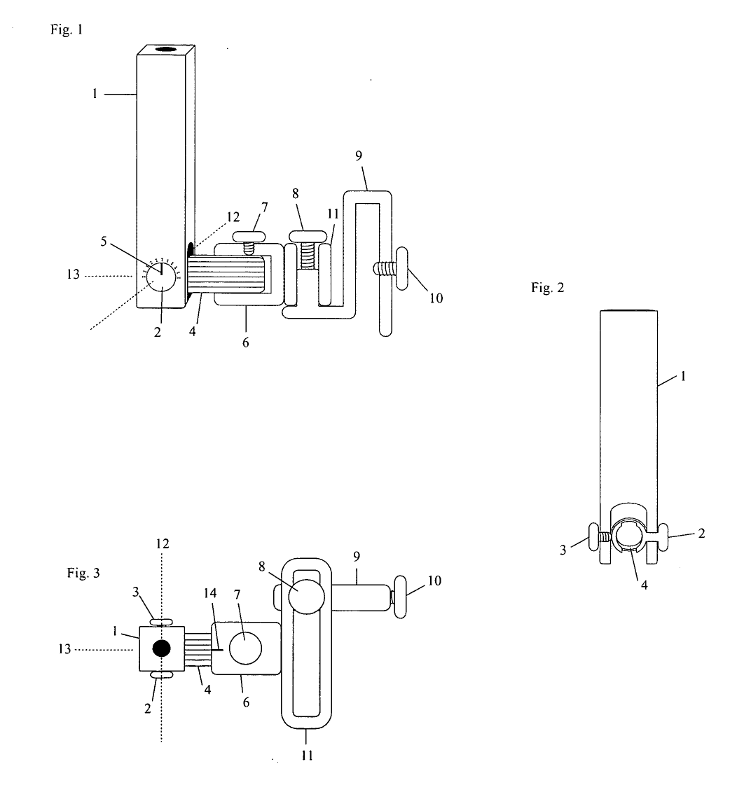

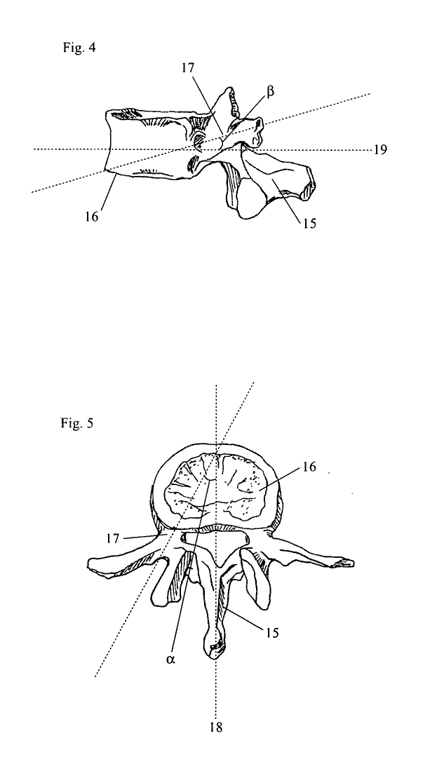

The side view of the apparatus shown in FIG. 1 demonstrates the drill guide 1 shaped as an elongated cube which is cylindrically hollow throughout its length to accommodate the drill bit or guide wire. FIG. 2 demonstrates that the drill guide 1 is slotted at its articulation with the barrel 4 to allow for accommodation of the barrel and to allow for rotation of the drill guide. FIG. 1 and FIG. 3 demonstrate that the drill guide 1 rotates in the axis of rotation 12 which is through the center of the drill guide angle knob 2 and is perpendicular to the barrel 4. The drill guide angle knob 2 is a process arising from the surface of the barrel 4 which has a cylindrical portion traversing through the drill guide 1 and ends in a disk-like shape on the outer surface of the drill guide. The drill guide angle knob 2 has a pointer mark 5 as seen in FIG. 1. The surface of the drill guide 1 immediately surrounding the drill guide angle knob 2 has calibrated degree markings corresponding to the circle of the drill guide angle knob. Aligning the pointer mark 5 of the drill guide angle knob 2 to the desired degree marking on the surface of drill guide 1 establishes the drilling angle in one plane. Tightening of the drill guide fastening screw 3 through the hole in the drill guide 1 onto the surface of the barrel 4 maintains the position of the drill guide via friction.

The barrel 4 is hollow and slotted at its articulating end with the drill guide 1 to allow for the passage of the drill bit or guide wire. At the other end, the barrel 4 fits lengthwise into the cylindrical slot of the body 6. FIG. 1 is a cross-section of this articulation. FIG. 3 is a view of this articulation from the top. Through this articulation, rotation and sliding of the barrel 4 is allowed. The barrel 4 rotates in the axis of rotation 13 which is through the long axis of the barrel and results in the concurrent rotation of the drill guide 1. Sliding of the barrel 4 within the cylindrical slot of the body 6 adjusts the distance of the drill guide 1 from the mounting clamp 9. The surface of the barrel 4 has calibrated degree markings along its length corresponding to the circle of the barrel. Aligning the pointer mark 14 located on the body to the desired degree marking on the barrel 4 establishes the drilling angle in the plane perpendicular to the previous manipulation. Tightening of the fastening screw 7 through a hole in the cylindrical slot of the body 6 onto the surface of the barrel 4 maintains this position of rotation and the distance of the drill guide 1 from the mounting clamp 9.

The loop portion of the body 11 allows for sliding along the mounting clamp 9; this motion is perpendicular to the sliding of the barrel 4 within the cylindrical portion of the body 6. Tightening of the loop screw 8 maintains this position.

The mounting clamp 9 is placed around the reference structure. Tightening of the clamp screw 10 through a hole in the mounting clamp and onto the reference structure maintains the position of the apparatus.

### Example of Use in Insertion of Pedicle Screw

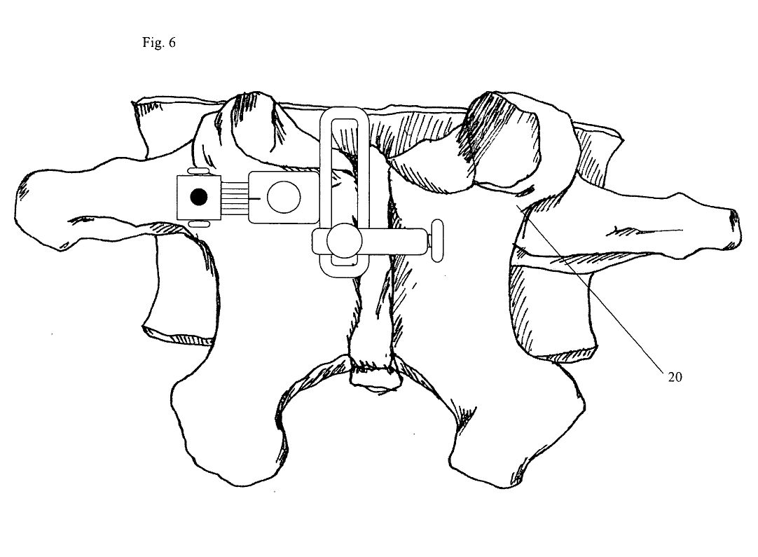

FIG. 4 is a side view of a vertebra. FIG. 5 is an axial view of a vertebra. The spinous process 15 of each vertebra is consistently midline and is oriented perpendicular to the vertebral body 16 and is the reference structures to which the drilling angle into the pedicle 17 will be derived. Preoperative imaging and/or anatomic knowledge is used for the following: In the sagittal plane, the medial/lateral angulation α of the pedicle with respect to a line parallel to the orientation of the spinous process 18 is determined. This will serve to set the angle of rotation of the drill guide 1 with respect to the barrel 4. In the axial plane, cephalad/caudal angulation β of the pedicle with respect to a line perpendicular to the orientation of the spinous process 19 is determined. This will serve to set the angle of rotation of the barrel 4 with respect to the cylindrical slot of the body 6.

The vertebra is surgically approached from the posterior. FIG. 6 is a view of the vertebra with the apparatus mounted (the apparatus is shown already localized to the entry point on the left pedicle). The mounting clamp 9 is placed on the spinous process 15 of the vertebra or on the interspinous ligaments (the ligaments that connect the adjacent spinous processes). After the mounting clamp is placed and tightened, the drill guide 1 is rotated to align the degree marking corresponding to α with the pointer mark of the drill guide angle knob 5. The drill guide fastening screw 3 is then tightened. The barrel 4 is rotated to align the degree marking corresponding to β with the pointer mark on the body 14. However, it is not tightened yet. The drill bit or guide wire is placed into and through the hole of the drill guide 1 and onto the surface of the bone. The drill bit or guide wire is manipulated to arrive at and contact the entry point of the pedicle screw 20 via sliding of the barrel 4 within the cylindrical slot of body 6 and sliding of the loop of the body 11 along the mounting clamp 9. Then, the barrel fastening screw 7 and the loop screw 8 are tightened to maintain the position. Drilling can then ensue through this guide system.

Thus an apparatus and method for drilling into the bone at the desired drilling angle has been shown and described above. It will be apparent that many changes, modifications, variations, and other uses and applications are possible and contemplated, and all such changes, modifications, variations, and other uses and applications which do not depart from the spirit and scope of the invention are deemed to be covered by the invention as is described in the Claims section.

I claim:

1. an apparatus which guides drilling into the bone at the desired drilling angle from an entry point on the surface of the bone comprising:
a. a mounting member which attaches the apparatus to a reference structure from which the desired drilling angle of entry in two perpendicular planes is derived;
b. rotationally linked members which allow the drill guide to rotate in two perpendicular axes, and the amount of rotation can be adjusted and fixed to maintain the drill guide angle;
c. and mechanisms to coincide the drill guide directly onto the entry point on the surface of the bone.

2. the apparatus claimed in 1 comprising:
a. a drill guide articulating with a cylindrical member such that the axis of rotation of the drill guide with respect to the cylindrical member is perpendicular to the long axis of the cylindrical member;
b. the aforementioned cylindrical member in turn articulating lengthwise with a congruent portion of the apparatus such that the axis of rotation of the cylindrical member with respect to the congruent portion of the apparatus is parallel to the long axis of the cylindrical member.

3. the apparatus claimed in 2 whereby rotations are adjustable to the desired angle by aligning calibrated markings on one member (corresponding to the degrees of rotation) with a reference mark on the other member, and the position of the rotation is maintained by turning of the fastening screw which exerts compressive friction and limits the movement of one member relative to another;

4. the apparatus claimed in 1 whereby the mounting member is comprised of a clamp with a screw to exert compressive friction onto the reference structure so that the position of the apparatus is held fixed.

5. the apparatus claimed in 1 coincides the drill guide onto the entry point on the surface of the bone by allowing sliding of the cylindrical member inside the congruent portion of the apparatus to adjust for distance in one plane while allowing sliding of the loop portion of the apparatus along the mounting member to adjust for distance in the perpendicular plane.
